(19) United States
(12) Patent Application Publication
(10) Pub. No.: US 2012/0046603 A1
(43) Pub. Date: Feb. 23, 2012

(54) HYPERBARIC DRESSING AND METHOD

(75) Inventor: Melvin Frederick Vinton, Cambridgeshire (GB)

(73) Assignee: Hitachi, Ltd.

(21) Appl. No.: 13/059,683

(22) PCT Filed: Aug. 14, 2009
(86) PCT No.: PCT/GB09/01987

§ 371 Date: Apr. 18, 2011

(30) Foreign Application Priority Data
Aug. 18, 2008 (GB) 0815078.1

(51) Int. Cl. A61M 37/00; A61M 35/00

(52) U.S. Cl. 604/24; 604/305; 604/290

(57) ABSTRACT

In a hyperbaric dressing a first fluid, such as oxygen, is deliverable between a fluid-impermeable layer impermeable to the first fluid and a fluid-permeable layer permeable to the first fluid. Edges of the fluid-impermeable layer and the fluid-permeable layer are sealed together and edges of the dressing are securable to a patient's skin surrounding a wound. Thus, when the first fluid is delivered, it can permeate through the fluid-permeable layer into a headspace between the dressing and the wound. A perforation is defined through the fluid-permeable layer and the fluid-impermeable layer for the passage of a second fluid, such as wound exudate. The perforation is open when a pressure in the headspace between the wound and the dressing is above a pre determined pressure and is closed when the pressure is below the predetermined pressure.

The present invention related to a hyperbaric dressing and methods of using a hyperbaric dressing.

### BACKGROUND OF THE INVENTION

It is known that a supply of oxygen to a wound or through the skin covering a wound can be used to promote healing and reduce scarring of damaged tissue. Typically, oxygen is absorbed by tissue fluids, thus improving the oxygen content of intercellular fluids and/or promoting metabolism and repair of the damaged tissue.

As such, there are numerous ailments which may benefit from the topical application of oxygen to damaged tissue, for example, osteomyelitis, tendon, ligament and cartilage damage, fractures, burns, scalds, necrotising fasciitis, such as pyoderma gangrenosum, pressure-induced decubitus (bed sores), venous and diabetic foot and leg ulcers, as well as cuts, abrasions and surgically-induced wounds and incisions.

In the healing process of non-infected wounds, low levels of exudate moisturising the skin surrounding a wound may be considered positive. When exudate becomes excessive or the wound becomes ‘chronic’ and non-healing or when infection becomes established, exudate may take on a different guise and has justifiably been termed ‘a wounding agent in its own right’ as it has the capacity to degrade growth factors. Excessive and particularly infected exudate from non-healing wounds may cause maceration to intact skin inhibiting the healing process. Mild maceration can be seen in the puffy whiteness to skin surrounding a wound when a ‘sticking plaster’ is removed.

With high exudating wounds (1-50 ml/24 hrs) dressings quickly becomes saturated, preventing access to oxygen and allowing maceration. Exudate flow is unpredictable in both timing and volume and is dependent on a number of patient-related conditions including the degree of mobility and (particularly with leg ulcers) the elevation of the wound. A conventional absorbent dressing becomes saturated with exudate, has no access to oxygen and may experience a ‘strikethrough’ where exudate seeps from the dressing to soil clothing etc.

Consequently, there is a desire to combine ideal wound healing conditions with exudate removal. To achieve this, it is desirable for wounds to be dressed and sealed from sources of external infection, have access to oxygen and moisture and have excessive exudate removed.

### SUMMARY OF THE INVENTION

The invention relates to a hyperbaric dressing and a method of use of a dressing as defined in the appended independent claims to which reference should now be made. Advantageous or preferred features are set forth in the dependent claims.

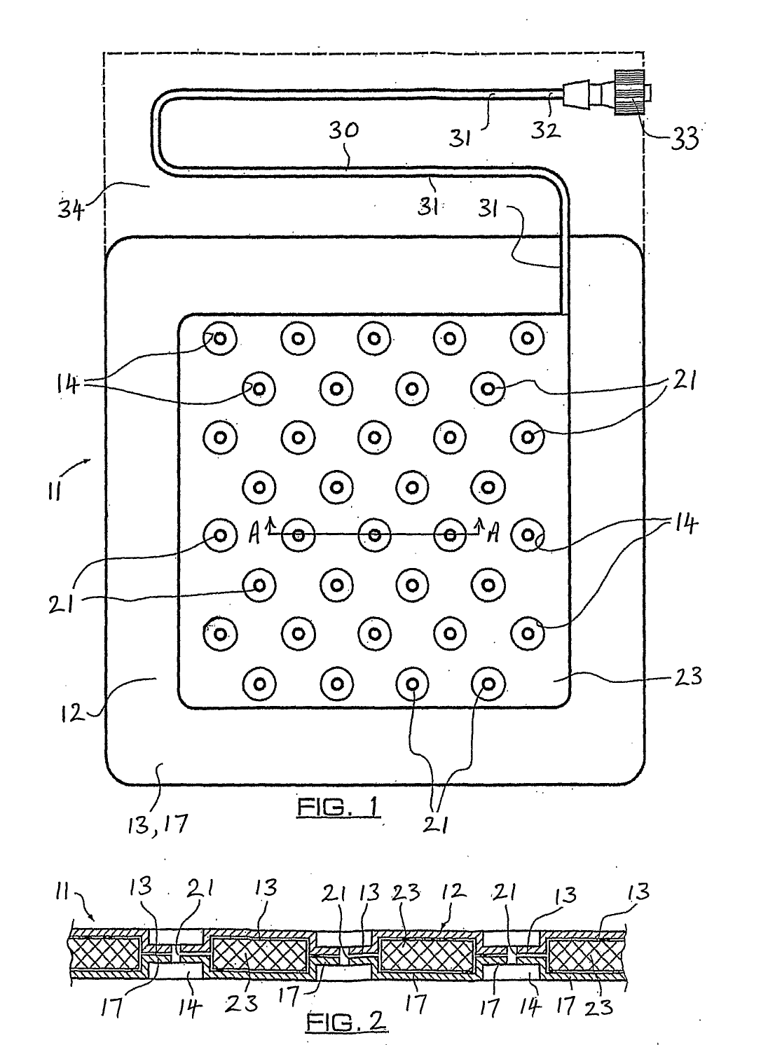

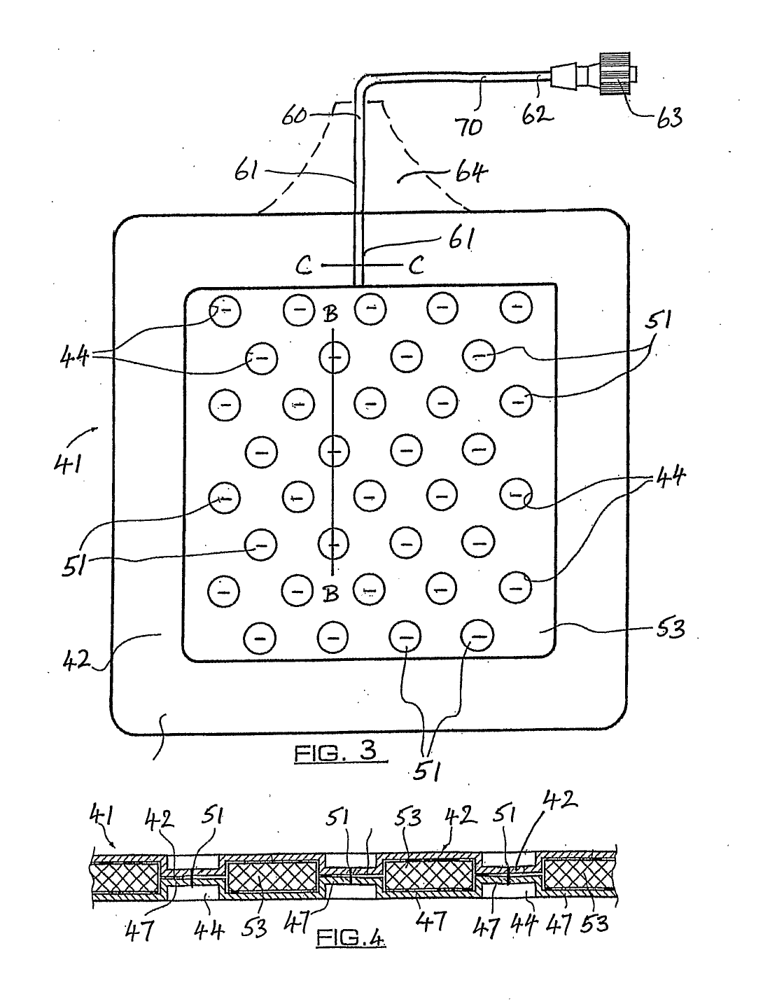

The applicant's UK patent application number GB-A-2412589, which is incorporated herein in its entirety, discloses a hyperbaric dressing that provides a means for locally supplying a wound with oxygen. This is illustrated in FIG. 1 (a plan view of the dressing) and FIG. 2 (a transverse section of the dressing). An upper layer 12 comprises a flexible film 13 that is oxygen impermeable and a lower layer 17 that is gas permeable. The upper and lower layers are sealed together around their peripheries to form a pouch. In between the permeable and impermeable layers is a porous material 23, which is gas permeable and has an array of circular apertures 14 extending through its thickness. The upper layer 12 and the lower layer 17 are joined together through the apertures 14. Holes 21 penetrate through both the upper and lower layers within the bounds of the apertures 14. An integral tube 30 is connectable to an oxygen source (not shown) through a connector 33, and allows oxygen to be supplied between the upper and lower layers. A self-adhesive layer (not shown) is attached to the lower layer of the dressing.

In use, the dressing is placed over a wound or damaged tissue (not shown) using the self-adhesive layer such that the lower layer 17 is nearest to the wound. Oxygen is supplied through the tube 30 between the upper layer 12 and lower layer 17, and then passes through the lower layer towards the wound. Consequently, oxygen flows in one direction towards the wound and any exudate produced by the wound can flow in the opposite direction through the holes 21 for removal.

Part of the structure of the dressing of the invention is similar to this prior art dressing, as follows. A hyperbaric dressing according to the present invention comprises a fluid-impermeable layer impermeable to a first fluid (such as oxygen for example), a fluid-permeable layer permeable to the first fluid for positioning over damaged tissue, and a perforation defined by the dressing to allow passage of a second fluid (such as exudate for example) through both the fluid-impermeable layer and the fluid-permeable layer.

The first fluid is deliverable between the fluid-impermeable and fluid-permeable layer and can pass through the fluid permeable layer. However, the invention is characterised in that the perforation has a closed state and an open state such that in the closed state the second fluid does not flow through the perforation and in the open state the second fluid can flow through the perforation. The perforation is open when pressure between the damaged tissue and the dressing is above a predetermined pressure and is closed when the pressure is below a predetermined pressure. In use, the dressing may thus provide optimal healing conditions for damaged tissue whilst allowing excessive fluid produced by the is damaged tissue to be removed. Damaged tissue may be sealed from sources of external infection, and have access to oxygen, and a beneficial amount of fluid may be maintained under the dressing.

The first fluid may comprise oxygen to aid in the healing of tissue. Alternatively or additionally, the first fluid may comprise other beneficial reagents such as cosmetic, anti-microbial agents, healing agents and pain-reducing agents, which may be administered constantly or periodically.

Advantageously, the dressing may remain in place over a period of time, for example several days or a week, without removing the dressing or disturbing the wound bed.

The second fluid may be wound exudate, a beneficial amount of which may be automatically maintained underneath the dressing whilst in use.

In a preferred embodiment the perforation comprises a slit, or cut. This may provide a simple and effective means for implementing a self-regulating perforation. Exudate wetting the slit may form a meniscus to restrict or seal the slit, which may then open when pressure builds in response to oxygen and exudate inflow in a self-regulating manner. Alternatively, the perforation may comprise any form of pressure valve, for example, a flap covering an opening. A plurality of perforations may be defined in the dressing, which are preferably distributed across an area of the dressing to allow an even flow of exudate, or to accommodate different exudate flows from different portions of a wound for example. The number and distribution of the perforations may be predetermined depending on the nature and size of the wound.

The length of the slit may be between 1 and 5 mm or preferably between 1.5 and 3.5 mm or particularly preferably approximately 2 mm. Such lengths have been shown to be particularly beneficial in the regulation of exudate removal. The slits may advantageously open when the predetermined pressure is between atmospheric pressure and the pressure of the supply of the first fluid, or preferably between 15 mmHg and 35 mmHg (2 kPa and 4.67 kPa) above atmospheric pressure.

Preferably, the perforation is defined within a membrane spanning a path for the second fluid to flow through the permeable layer and the impermeable layer. The fluid-permeable layer and the fluid-impermeable layer may be sealed together around the periphery of the membrane. The membrane may be formed by the fluid-permeable layer, the fluid-impermeable layer, a separate membrane layer, or by any combination of these layers overlying each other or sealed together.

For example, a separate membrane layer may extend across the whole dressing or may be present only in the region of the path for the flow of the second fluid. If such a separate membrane layer is used, its thickness and materials properties may be selected to enhance the performance of the perforation, without affecting the performance of the fluid-permeable and the fluid-impermeable layers.

The thickness and materials properties of the membrane and the dimensions and shape of the perforation may be predetermined such that the perforation is open to allow flow of the second fluid when pressure between the dressing and the damaged tissue is above the predetermined pressure. Advantageously, such features of the dressing may be manipulated depending on the nature of the damaged tissue to which the dressing is to be applied. Some wounds may exude greater amounts of exudate or vary in the viscosity of the exudate. For example, burns are often characterised by having a discharged fluid which is protein-rich plasma and is usually produced in great quantities.

In a preferred embodiment, the fluid-impermeable layer and/or the fluid-permeable layer comprise a plastics material. For example, the fluid-impermeable layer may comprise polyethylene or, alternatively, polyurethane.

Preferably, the thicknesses of the fluid-impermeable layer and/or the fluid-permeable layer are 0.05 mm to 1.00 mm or particularly preferably 0.1 mm to 0.5 mm.

The dressing may comprise a porous layer between the fluid-permeable layer and the fluid-impermeable layer to help maintain separation of the fluid-permeable and fluid-impermeable layers. The porous layer may, for example, comprise an open-cell foam. Apertures may be defined through the porous layer, through which the fluid-impermeable layer and the fluid-permeable layer are sealed together.

In a preferred embodiment, the dressing further comprises an adhesive layer for application of the dressing over the damaged tissue. The adhesive layer is preferably arranged such that when it is applied to the damaged tissue it forms a seal to allow pressurisation of a space (or headspace) between the damaged tissue and the dressing and so that exudate may exit through the perforations. To achieve this, the adhesive layer may be located on or near the peripheral edge of the dressing.

The peripheral edges of the fluid-permeable and fluid-impermeable layers are preferably secured together to form a pouch to aid in directing the first fluid through the fluid-permeable layer towards the damaged tissue and allow even distribution of the first fluid across the damaged tissue.

Unlike a conventional absorbent dressing, which remains saturated with exudate, in a dressing embodying the invention the headspace (between the dressing and the damaged tissue) may be constantly refreshed with humidified oxygen. Bacteria commonly found in infected leg ulcers are anaerobic and cannot survive in an oxygen rich atmosphere. Controlling infection is particularly important in non-healing wounds particularly with long term patients who may have resistance to antibiotics.

The dressing may be used as a primary or secondary dressing. This is particularly relevant as clinicians and patients have preferences. Pre-clinical evaluations reveal no significant reduction in achieving an oxygen-rich headspace (in between the damaged tissue and the dressing) when a separate primary dressing is used (for example, when a conventional absorbent dressing is used beneath a dressing embodying the invention) as these dressings are highly permeable and oxygen, is readily absorbed.

The invention may thus provide a hyperbaric dressing which accomplishes the same function as known oxygen chambers, bags and the like known in the art but at much less expense. Also, it may be more easily positioned upon a patient and is easily removable after use. Furthermore, it is readily disposable and requires no sterilization after use. During use, a patient can enjoy substantially full mobility, particularly if a small portable oxygen generator or cylinder is available, is whilst employing any suitable existing type of absorbent dressing over the hyperbaric dressing for absorbing exudate that flows through the perforations.

According to another aspect of the invention, there is provided a method of treating a human or animal to assist the healing of damaged tissue. A hyperbaric dressing as described above is applied to damaged tissue and the dressing is supplied with a first fluid.

In another aspect of the invention, there is provided a method for cosmetically treating a human or animal to reduce the existence and/or visibility of scar tissue. A hyperbaric dressing, as described above, is applied to the scar tissue and the dressing is supplied with a first fluid.

### SPECIFIC DESCRIPTION OF THE PREFERRED EMBODIMENTS

Embodiments of the invention will now be described, by way of example, with reference to the accompanying drawings in which:

FIG. 1 (prior art) is a plan view of a hyperbaric dressing in use;

FIG. 2 (prior art) is a partial transverse section of the dressing shown in FIG. 1;

FIG. 3 is a plan view of a hyperbaric dressing according to a first embodiment of the present invention;

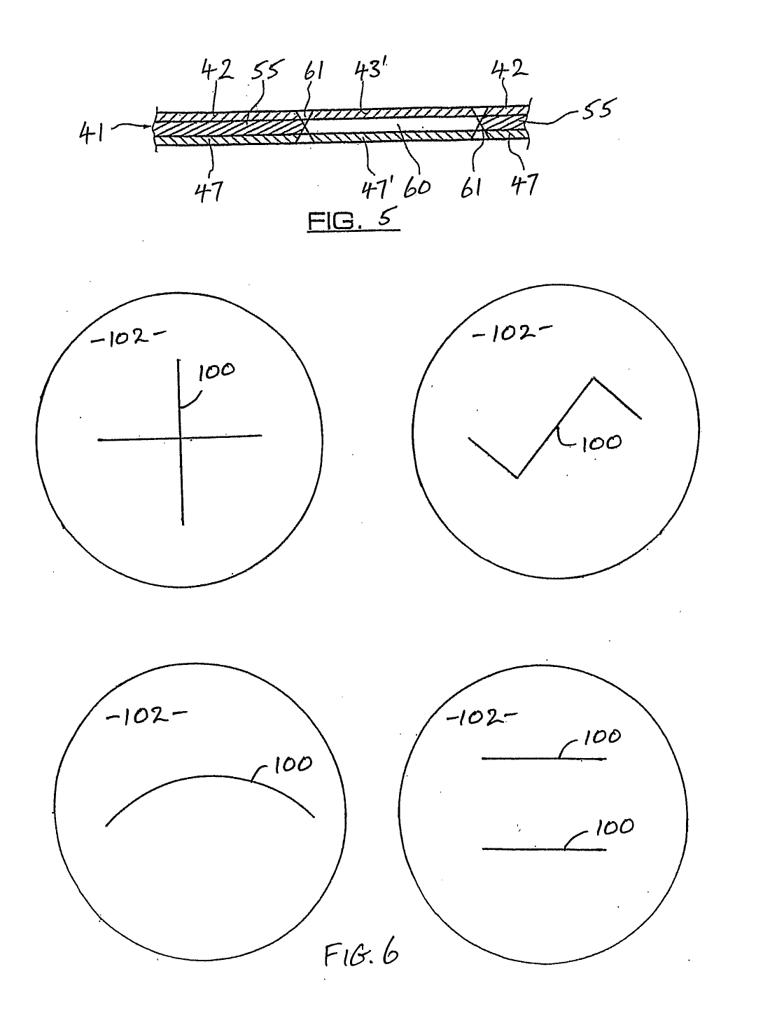

FIG. 4 is a partial transverse section, on B-B, of the dressing shown in FIG. 3;

FIG. 5 is a partial transverse section, on C-C, of the dressing shown in FIG. 3;

FIG. 6 shows four examples of perforations in the form of slits of various lengths and shapes;

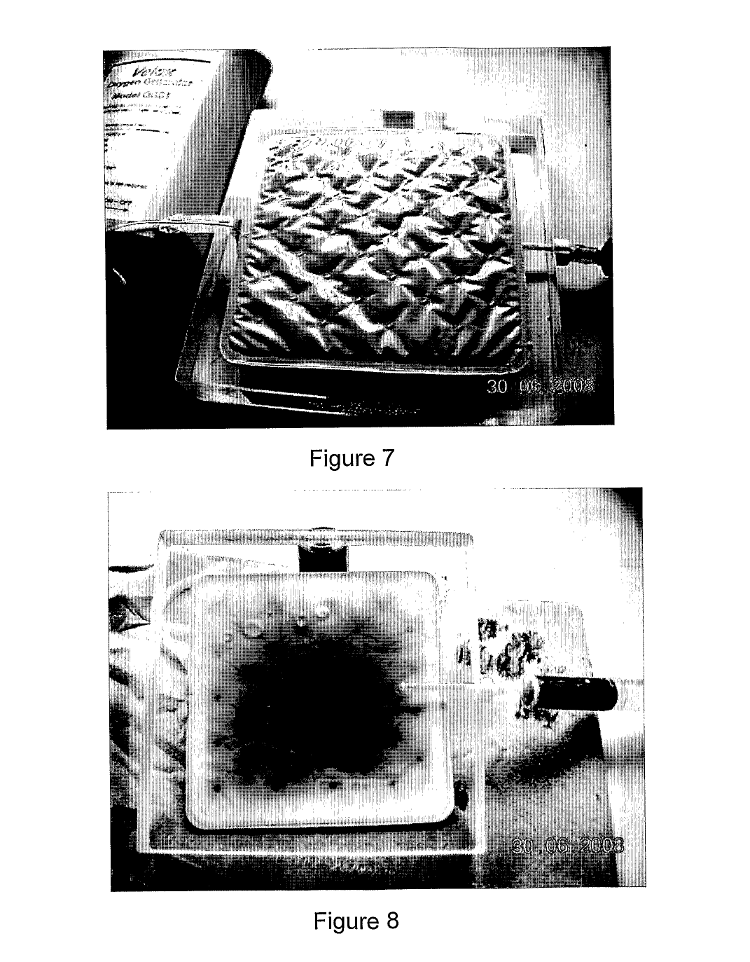

FIG. 7 shows a dressing embodying the invention placed on a wound model and supplied with oxygen;

FIG. 8 shows the dressing of FIG. 7 supplied with 5 ml of a model exudate;

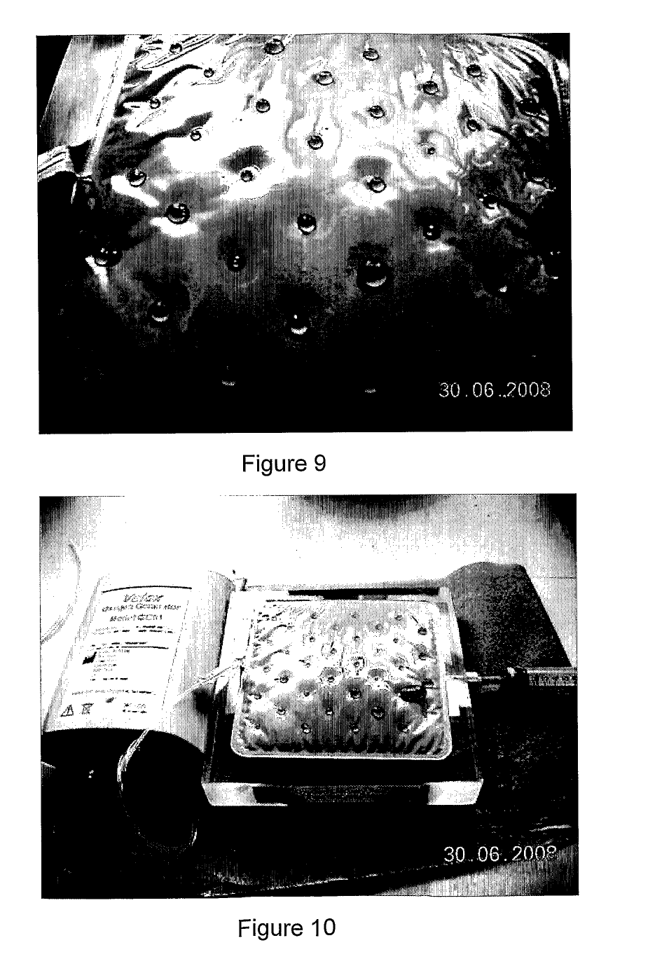

FIG. 9 is the dressing of FIGS. 7 and 8 when the apparatus was turned to normal use position;

FIG. 10 shows the dressing of FIGS. 7 to 9 at 30 minutes;

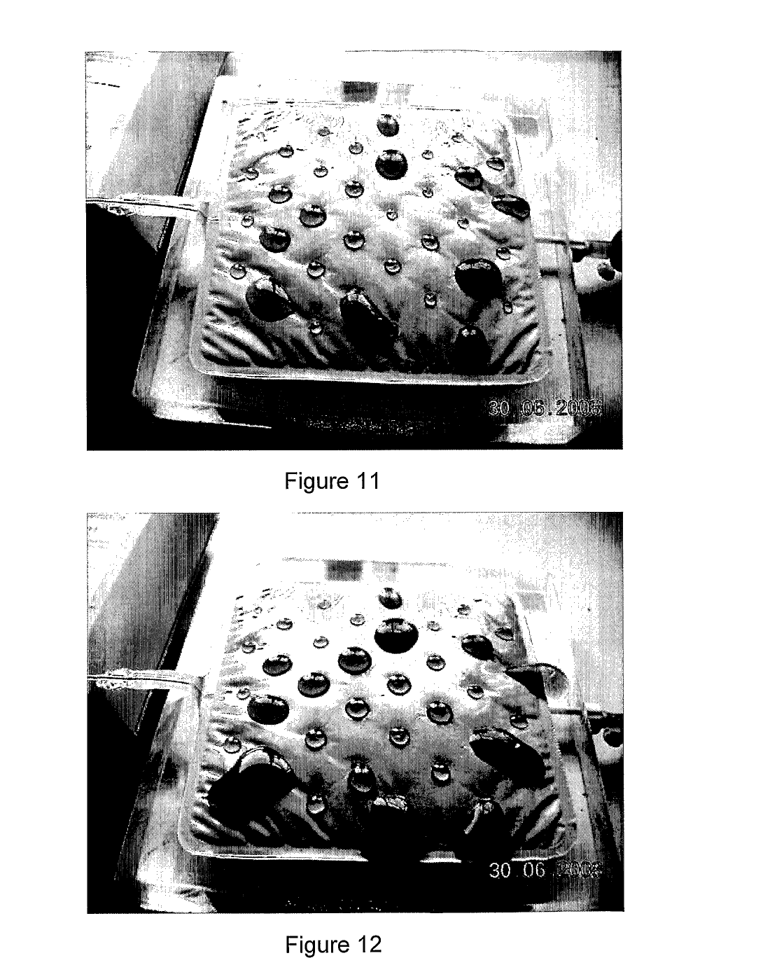

FIG. 11 shows the dressing of FIGS. 7 to 10 at 45 minutes after the addition of 5 ml of a model exudate;

FIG. 12 shows the dressing of FIGS. 7 to 11 at 60 minutes after the addition of 5 ml of a model exudate; and

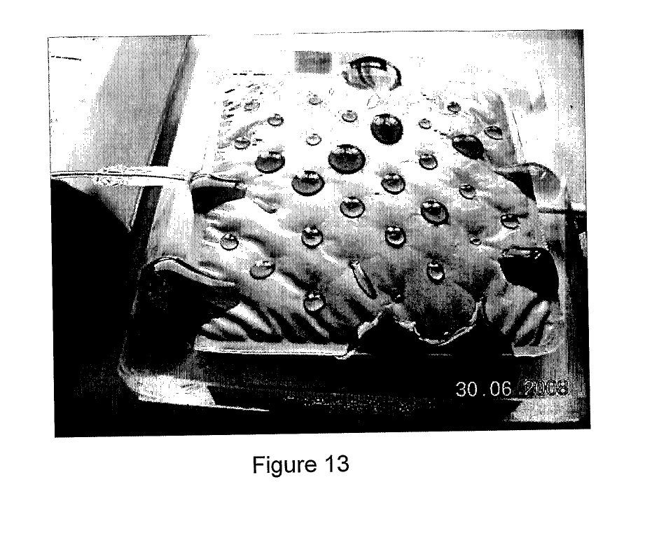

FIG. 13 shows the dressing of FIGS. 7 to 12 at 90 minutes.

A hyperbaric dressing embodying the invention will now be described with reference to FIGS. 1 to 13.

The overall structure is similar to that described in the prior art with reference to FIGS. 1 and 2, as discussed above.

As shown in FIGS. 3 and 4, a dressing 41 comprises a first, fluid-impermeable layer 42. This layer is made from a plastics material such as a polyethylene film and is impermeable to gaseous oxygen. A second, fluid-permeable layer 47 is provided by a gas-permeable, and specifically oxygen-permeable, sheet of material, such as that sold under the Trade Mark “CAPLA”. Each layer is typically in the range of 0.05 mm to 1 mm thick and most preferably in the range of 0.1 mm to 0.5 mm thick. In between the impermeable and permeable layers is a thicker sheet 53 of an open-cell foam material, which is porous and has a substantially regular array of circular apertures 44 extending through its thickness. The first layer 42 and second layer 47 are, using a suitable tool (not shown), heat-sealed together through the circular apertures 44 to form a substantially planar membrane within each aperture. Simultaneously or subsequent to such heat sealing, perforations in the form of slits 31 are formed through the resulting membranes, as shown in particular in FIG. 4.

FIG. 3 shows a substantially regular array of apertures 44 in which slits 51 are is situated. The number of apertures and slits may vary depending on such factors as the size of the wound, the required amount of exudate removal and the predetermined pressure at which the slits are required to open. The predetermined pressure is above atmospheric pressure and is typically above 10 mmHg (1.33 kPa). Preferably, the pre-determined pressure at which the slits open to allow exudate to flow is 15 mmHg to 35 mmHg (2.00 kPa to 4.67 kPa).

The length and shape of the slits may vary depending on a variety of factors such as the nature of the surface to which the dressing is applied, the size of the wound, how much exudate is produced and the viscosity of the fluid. Some variations in slit shape and/or size are shown in FIG. 6, which shows four slit configurations 100 defined in circular membranes 102. Altering the size and/or shape of the slits may enable manipulation of the pre-determined pressure at which the slits open, and the volume of exudate flow that can be accommodated. The size and shape of the slits may also be predetermined in response to the type and amount of fluid to be delivered to the dressing.

As with the prior art dressing discussed above with reference to FIGS. 1 and 2, self-adhesive layers may be employed to allow application of the dressing. The dressing may be applied by removing a peel-off layer to expose the self-adhesive layer. The adhesive layer may be positioned around the periphery of the fluid-permeable layer such that the space between the wound and the dressing may become pressurised. These features are not shown in FIGS. 1 to 6.

In this embodiment of the invention the gas delivery arrangement is provided by a conduit comprising an integral tube or cannula 60 formed (within the dressing) from adjacent portions of the fluid-impermeable layer 42 and the fluid-permeable layer 47, as shown in detail in FIG. 5.

Formation of the integral tube 60 is carried out by providing a pair of spaced, sealing weld lines 61, between the fluid-impermeable layer 42 and the fluid-permeable lower layer 47. The integral tube 60 is thus formed between respective portions 43′ and 47′ of the layers which are not sealed together, and is secured to a conventional external tube 70.

In use, the end 62 of the tube 70 remote from the dressing 41 can be connected to an oxygen source (not shown) by means of a connector 63. Oxygen is delivered at a pressure greater than atmospheric pressure. The resulting oxygen pressure inside the open-cell foam sheet 53 forces oxygen through the gas-permeable layer in one direction and on to or over the wound. Other fluids such as healing agents and pain relievers may also be delivered through the same means.

The tube may not be integral to the dressing and delivery may be through a separate tube, which may be connected and disconnected to the dressing. Such a means for delivering a fluid may be connectable into a socket of the dressing as shown in FIG. 7 of UK Patent Application No. GB-A-2412589.

It is noted that embodiments of the present invention also extend to those described in UK patent application GB-A-2412589 with the beneficial modification that holes are replaced by perforations, such as slits, that open when the pressure is above a predetermined pressure and close when the pressure is below a predetermined pressure.

All materials and processing techniques should preferably be in compliance with relevant regulatory requirements. An embodiment of the invention may comprise materials such as the following:
1. Tube/cannula (60): Part No 800/100/280. Supplier: Smiths medical. Length: 1000 mm±10 mm.
2. Tube connector (63) (Female Luer fitting): Part No 65206. Supplier: Qosina.
3. Top layer (Fluid impermeable sheet (43)). Part No L340. Supplier: Braun Hospicare.
4. Open cell foam (53). Part No 4200. Supplier: Calligan foam.
5. Lower layer (Fluid permeable layer (47)): PE film. Part No BF-633 35 gm/m². Supplier: TREDEGAR film products.
6. Self-Adhesive strip 8 mm: Part No 1522 3M. Supplier: 3M medical tape division.

An experiment was performed to simulate an embodiment of the present invention, in use.

The slit dimensions for advantageous dressing performance such as maintaining oxygen pressure and removal rates of exudate were investigated in the experiment using the following process:

It is understood that 75% of venous ulcer wound exudate shows a viscosity of 8 mPa/s or less. The standard simulator for wound exudate, that is accepted for trials, is xanthan gum, which is a polysaccharide having E number 145 and used to increase food thickness. Exudate may be modeled with dilute aqueous solutions of 0.1% w/w concentration. This solution is opaque and a food (blue) colouring was added for clarity.

An apparatus was constructed where the dressing was placed on a Perspex apparatus model and connected to an oxygen supply with pressure measured using a water manometer. The oxygen supply was connected to the dressing while simulated exudate flowed to fill the headspace beneath the dressing such that the exudate was in contact with the lower layer of the dressing, as in normal use conditions.

Oxygen flow was constant at approximately 13 ml/hour. The active area of the dressing (through which oxygen is delivered) was 98 cm² with the combined area of the 36 membranes (in which slits are defined) being 2 cm².

A range of different slit configurations and lengths were tried. Evaluations continued using slits as described below.
1. Each 5 mm diameter membrane of the dressing was slit centrally along a length of 2 mm.
2. The dressing was placed on the wound model and oxygen was allowed to flow until a ‘cushion’ appearance was seen as oxygen fills and diffuses through, raising it from the model wound surface (FIG. 7).
3. 5 ml of exudate was applied to the lower (wound) side of the dressing, which spread across the enclosed headspace beneath the dressing (FIG. 8).
4. The apparatus was turned to normal use position (horizontal; the apparatus had been tilted as shown in FIG. 8 when the exudate was injected into the headspace). The dressing resisted the additional (exudate) volume by allowing the exudate to leak through the slits (FIG. 9), driven by the pressure in the headspace.

The oxygen supply remained connected and FIGS. 10, 11, 12 and 13 illustrate the situation at 30 mins, 45 mins (after 5 ml of exudate was added), 60 mins (after 5 ml of exudate was added) and 90 mins respectively.

As can be observed in these Figures, as oxygen continued to flow and more exudate was added, corresponding exudate outflow was seen. Under normal conditions this would be ‘wicked’ up by an absorbent outer dressing, which may be changed without disturbing the dressing.

2 mm slits centrally across each of the apertures or membranes allows typical wound exudate outflow whilst maintaining headspace oxygen pressure. Although these evaluations were made on dressings with no outer supporting bandaging or absorbent retaining pads, pre-clinical evaluations reveal that the only effect of supporting or tubular bandage is to flatten and restrain the dressing reducing the volume of the headspace. Diffusion levels of oxygen remain unaffected, although the reduced dressing headspace volume increases the oxygen gradient in the headspace.

1. A hyperbaric dressing comprising:
a fluid-impermeable layer impermeable to a first fluid;
a fluid-permeable layer permeable to the first fluid, for positioning over damaged tissue; and
a perforation defined by the dressing to allow passage of a second fluid through the fluid-permeable layer and the fluid-impermeable layer;
in which the first fluid is deliverable between the fluid-impermeable layer and the fluid-permeable layer such that in use the first fluid passes through the fluid permeable layer, and in which the perforation has a closed state and an open state, such that in the closed state the second fluid does not flow through the perforation and in the open state the second fluid flows through the perforation, the perforation being open when pressure between the damaged tissue and the dressing is above a predetermined pressure and the perforation being closed when the pressure is below a predetermined pressure.

2. A dressing according to claim 1, in which the perforation is in the form of a slit.

3-4. (canceled)

5. A dressing according to claim 1, in which the predetermined pressure is between 15 mmHg and 35 mmHg, above atmospheric pressure.

6. A dressing according to claim 1, in which the perforation is defined within a membrane spanning a path for the second fluid to flow through the fluid-permeable layer and the fluid-impermeable layer.

7. A dressing according to claim 6, in which the fluid-impermeable layer and the fluid-permeable layer are sealed together around a periphery of the membrane.

8. A dressing according to claim 6, in which the membrane is formed by one or more of the fluid-permeable layer, the fluid-impermeable layer, and a membrane layer.

9. A dressing according to claim 8, in which the membrane comprises in-register portions of the fluid-impermeable layer and the fluid-permeable layer sealed together.

10. A dressing according to claim 9, in which the thickness and materials properties of the membrane, and the dimensions and shape of the perforation, are predetermined such that the perforation is open to allow flow of the second fluid when the pressure between the dressing and the damaged tissue is above the predetermined pressure.

11. A dressing according to claim 1, in which the fluid impermeable layer comprises a plastics material.

12. (canceled)

13. A dressing according to claim 1, in which the fluid permeable layer comprises a plastics material.

14. A dressing according to claim 1, in which the fluid impermeable layer and/or the fluid-permeable layer are 0.05 mm to 1.00 mm thick.

15. A dressing according to claim 14, in which the fluid-impermeable layer and/or the fluid-permeable layer are 0.1 mm to 0.5 mm thick.

16. A dressing according to claim 1, which further comprises a porous layer between the fluid-permeable and fluid-impermeable layer, the porous layer being permeable to the first fluid.

17. A dressing according to claim 16, in which an aperture is defined through the porous layer, such that the path for the second fluid to flow through the fluid-permeable layer and the fluid-impermeable layer passes through the aperture.

18. (canceled)

19. A dressing according to claim 1, which further comprises an adhesive layer for application of the dressing over the damaged tissue.

20. (canceled)

21. A dressing according to claim 1, in which peripheral edges of the fluid-impermeable layer and the fluid-permeable layer are secured together to form a pouch.

22. A dressing according to claim 1, in which the first fluid comprises oxygen.

23. A dressing according to claim 1, in which the second fluid comprises exudate.

24. (canceled)

25. A dressing according to claim 23, further comprising an absorbent or compression outer bandage or dressing for absorbing the exudate, or a vacuum means for the removal of the exudate.

26. A method of treating a human or animal tissue, comprising the steps of:
applying a hyperbaric dressing to the tissue, in which the hyperbaric dressing comprises:
a fluid-impermeable layer impermeable to a first fluid;
a fluid-permeable layer permeable to the first fluid, for positioning over the tissue; and
a perforation defined by the dressing to allow passage of a second fluid through the fluid-permeable layer and the fluid-impermeable layer;
in which the first fluid is deliverable between the fluid-impermeable layer and the fluid-permeable layer such that in use the first fluid passes through the fluid permeable layer, and in which the perforation has a closed state and an open state, such that in the closed state the second fluid does not flow through the perforation and in the open state the second fluid flows through the perforation, the perforation being open when pressure between the tissue and the dressing is above a predetermined pressure and the perforation being closed when the pressure is below a predetermined pressure; and
supplying the dressing with a fluid.

27. (canceled)
